(19) United States
(12) Patent Application Publication
(10) Pub. No.: US 2015/0025369 A1
(43) Pub. Date: Jan. 22, 2015

(54) HOUSING FOR THE OCT PROBE, OCT PROBE ASSEMBLY, AND A METHOD OF MAKING SUCH ASSEMBLY

(71) Applicant: Corning Incorporated, Corning, NY (US)

(72) Inventors: Venkata Adiseshaiah Bhagavatula, Big Flats, NY (US); John McKenna Brennan, Vero Beach, FL (US); Woraphat Dockchoorung, Horseheads, NY (US); Klaus Hartkorn, Painted Post, NY (US); Mark Alan McDermott, Prattsburgh, NY (US); Amorn Runarom, Samutsakhon (TH); Daniel Max Staloff, Rochester, NY (US)

(21) Appl. No.: 14/313,087

(22) Filed: Jun. 24, 2014

Related U.S. Application Data
(60) Provisional application No. 61/847,288, filed on Jul. 17, 2013.

(51) Int. Cl. A61B 5/00

(52) U.S. Cl. CPC A61B 5/0073
USPC 600/425; 29/527.1

(57) ABSTRACT

According to some embodiments a housing for the OCT comprises: (a) a tubular body with an inner diameter of less than 5 mm (for example less than 2 mm, and in some embodiments not greater than 1.5 mm), a first end, a second end; and a window formed in the tubular body closer to the second end than to the first end, displaced from the second end, and framed by a portion of the tubular body, wherein the window has a width w. According to some embodiments, 0.05 mm<w<8 mm.

### CROSS-REFERENCE TO RELATED APPLICATIONS

This application claims the benefit of priority under 35 U.S.C. §119 of U.S. Provisional Application Ser. No. 61/847288 filed on Jul. 17, 2013 the content of which is relied upon and incorporated herein by reference in its entirety.

### BACKGROUND

The disclosure relates generally to OCT probes, and more particularly to OCT probe assemblies and housing for OCT optical probe component.

No admission is made that any reference cited herein constitutes prior art. Applicant expressly reserves the right to challenge the accuracy and pertinency of any cited documents.

### SUMMARY

One embodiment of the disclosure relates to a housing for the OCT probe. The housing comprises:
(a) a tubular body with an inner diameter of less than 5 mm (for example 2 mm or less, and in some embodiments not greater than 1.5 mm), a first end, a second end; and
(b) a window formed in the tubular body closer to the second end than to the first end, displaced from the second end, and framed by a portion of the tubular body, wherein the window has a width w.

According to some embodiments, 0.05 mm<w<8 mm, for example, 0.05 mm<w<5 mm, or 0.1 mm<w<3 mm, 0.5 mm<w<2.5 mm, or 0.5 mm<w<2 mm. According to some embodiments, the tubular body includes at least one surface that has RMS surface roughness of ≦5 μm. According to some embodiments, the tubular body has at least one surface with the coefficient of friction ≦0.3.

According to some embodiments, an OCT probe assembly comprises:

(i) a micro optic component including
(a) a light transmissive rod having a first end, a second end, and a central axis;
(b) a reflective surface situated on the second end and slanted with respect to the central axis;
(c) a refractive surface adjacent to the second end; and

(ii) a housing surrounding the micro optic component; the housing having
a) a tubular body, and
b) a window formed in the tubular body and situated at least 0.2 mm away from end of the tubular body nearest the window, the window being framed by a portion of the tubular body, and is situated over the refractive surface.

For example, the a window may be situated at 0.5 mm, at least 1 mm, at least 1.5 mm, or at least 2 mm away from end of the tubular body nearest the window.

An additional embodiment of the disclosure relates an OCT probe assembly comprises:

(i) a unitary micro optic component including
(a) a light transmissive rod having a first end, a second end, and a central axis;
(b) a reflective surface situated on the second end and slanted with respect to the central axis;
(c) a raised lens element situated on the rod and being integral thereto, the lens element being adjacent to the second end, and to the reflective surface, the lens element having a curved refractive surface with at least one radius of curvature r1 wherein
   100 μm<r1<5000 μm, and a thickness t where 100 μm<t<3000 μm; and

(ii) a housing surrounding the micro optic component; the housing having
(a) a tubular body with a first end, and a second end adjacent to the second end of the rod, and
(b) a window formed in the tubular body near the second end of the tubular body, the window being framed by a portion of the tubular body, wherein the window is situated over the lens element and has a width w, where 50 μm<w<5000 μm (e.g., 50 μm<w<2000 μm).

According to some embodiments a method of making an OCT probe assembly comprises at least the following steps:

(i) providing a unitary micro optic component (including at least a lens element and a light transmissive rod), a fiber mount, and a fiber situated on or in the fiber mount and optically coupled to the micro optic component;

(ii) providing a housing having a tubular body with a bore situated therein, and a window formed in the tubular body;

(iii) inserting the unitary micro optic component and the fiber into the housing, such that the micro optic component and the fiber enter one of the apertures of the tubular body and is slid through at least a portion of the bore;

(iii) applying an adhesive material through the window into the bore;

(iv) sliding the micro optic component inside the bore such that
(a) the motion of the micro optic component spreads the adhesive material between the optic component and the bore surface, thus at least filling with the adhesive material the void situated between at least a portion of surface of the micro optic component and the surface of the bore, and
(b) the lens element is positioned in the window.
   According to some embodiments the method includes a step of sealing or plugging the second aperture of the tubular body (e.g., with an adhesive, or attaching a plug to the second over the second aperture of the tubular body).

An additional embodiment of the disclosure relates a method of making an OCT probe assembly comprising:

(i) providing a unitary micro optic component including
(a) a light transmissive rod having a first and a second end and a central axis;
(b) a reflective surface situated on the second end and slanted with respect to the central axis;
(c) a raised lens element situated on the rod and being integral thereto, and adjacent to the second end and to the reflective surface, the lens element having a curved refractive surface;
(d) a fiber mount adjacent to the rod and formed integral thereto'

(ii) inserting the unitary micro optic component into a housing having a tubular body with a bore, and a window formed in the tubular body and framed by a portion of the tubular body, such that the micro optic component enters through an entrance (i.e., first) aperture of the tubular body, and is slid through the bore such that at least the reflective surface and the lens element exit a second aperture of the tubular body;

(iii) applying an adhesive material through the window into the bore;

(iv) sliding the micro optic component back through the second aperture toward the first aperture such that (i) the motion of the micro optic component spreads the glue between the optic component and the bore surface, thus at least filling a void between the at least a portion of at least one surface of the micro optic component and the bore surface;

(v) positioning the lens element in the window; and

(vi) sealing or closing the second aperture of the tubular body.

Additional features and advantages will be set forth in the detailed description which follows, and in part will be readily apparent to those skilled in the art from the description or recognized by practicing the embodiments as described in the written description and claims hereof, as well as the appended drawings.

It is to be understood that both the foregoing general description and the following detailed description are merely exemplary, and are intended to provide an overview or framework to understand the nature and character of the claims.

The accompanying drawings are included to provide a further understanding, and are incorporated in and constitute a part of this specification. The drawings illustrate one or more embodiment(s), and together with the description serve to explain principles and operation of the various embodiments.

### BRIEF DESCRIPTION OF THE DRAWINGS

FIG. 1 illustrates a housing of an OCT probe including an OCT probe assembly situated in an inflatable balloon and an inner lumen, and a torque tube;

FIGS. 2A-2C illustrate one embodiment of a housing for an OCT probe component;

FIGS. 3A-3C illustrate another embodiment of a housing for an OCT probe component;

FIGS. 4A and 4B illustrate one embodiment of OCT probe assembly;

FIGS. 5A and 5B illustrate another embodiment of OCT probe assembly;

FIG. 6A illustrates schematically an embodiment of the OCT probe component including an exemplary refractive lens with positive optical power;

FIG. 6B illustrates schematically another embodiment of the OCT probe component including an exemplary lens; and

FIG. 7 illustrates an exemplary stainless steel coiled wire torque tube attached to one embodiment of the housing for an OCT probe component.

### DETAILED DESCRIPTION

In optical coherence tomography (OCT) imaging information about biological tissues can obtained by medical scanning done inside a living body, by utilizing an OCT probe 5 that contains a small optical probe component 20 (also referred to herein as miniature optic sensor, or a micro optic component 20) situated within the OCT probe assembly 10. The small optical probe component 20 images light provided by an optical fiber 21 onto the tissues, and collects the light scattered back by the tissues. For example the an OCT probe 5 including an inflatable balloon 8 and an OCT probe assembly 10 containing the small optical probe component 20 coupled to the optical fiber 21 is inserted inside the body, for example through the blood vessels or gastro intestinal tract, to obtain an image of the inside surfaces of the tissues such as blood vessels, or tissues of the intestinal tract.

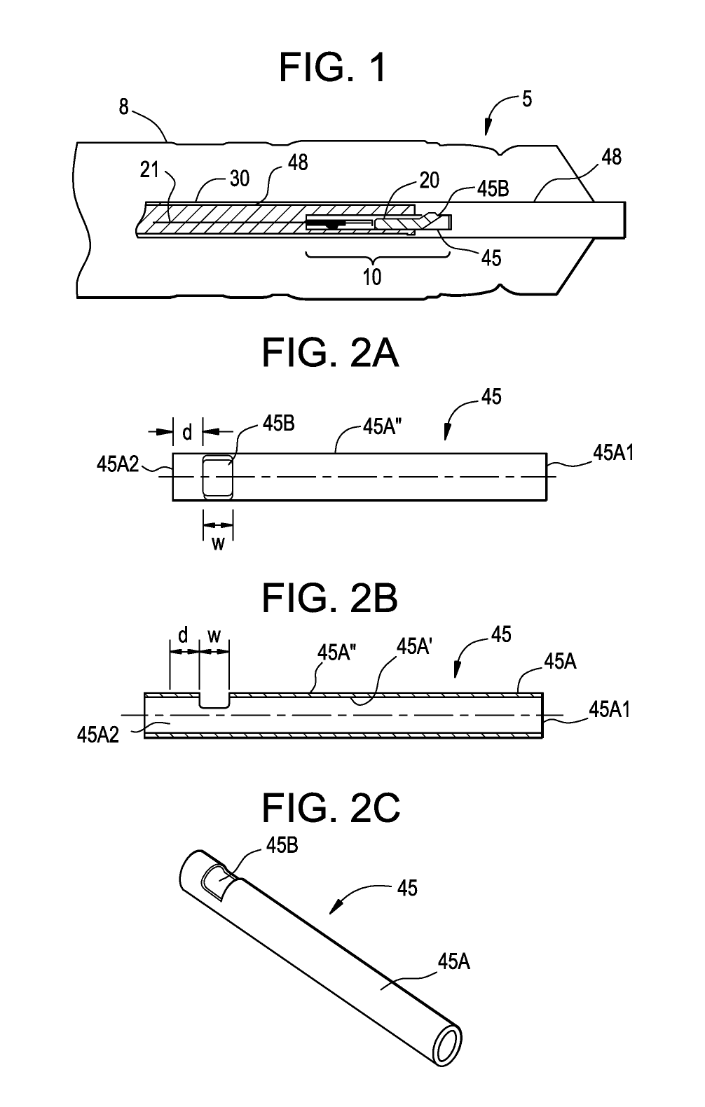

More specifically the OCT probe assembly 10 moves inside a body to obtain sub-surface 3D information of tissues. Light scattered back from the tissues (at different depths) is monitored using interferometric techniques, resulting in 3D scan of the tissues. The 3D scan is achieved by rotating the optical probe component 20 and its housing 45 at high speeds (for example greater than 1000 rpm, and preferably in the range of 3000 rpm-12000 rpm) in a controlled fashion. This rotation is achieved, for example, by using rotation/ translation device 30, for example, a stainless steel coiled wire torque tube that is attached to the optical probe component 20, and/or optical fiber 21, or to the housing 45. The rotation/translation device 30 such as stainless steel coiled wire torque tube 30 and the OCT probe assembly 10 that includes the optical probe component 20 and its housing 45 are then threaded through a close fitting transparent tube (e.g., made of polymer) referred to as an inner lumen 48. During OCT device operation, the OCT probe assembly 10 and the stainless steel coiled wire torque tube 30 rotate inside the inner lumen 48, and the inner lumen 48 protects the tissues from contact with the rotating probe assembly 10. A schematic of an OCT probe 5 including a portion of the torque tube, and the OCT probe assembly 10 situated in an inflatable balloon 8 is illustrated, for example, in FIG. 1.

Various embodiments will be further clarified by the following examples.

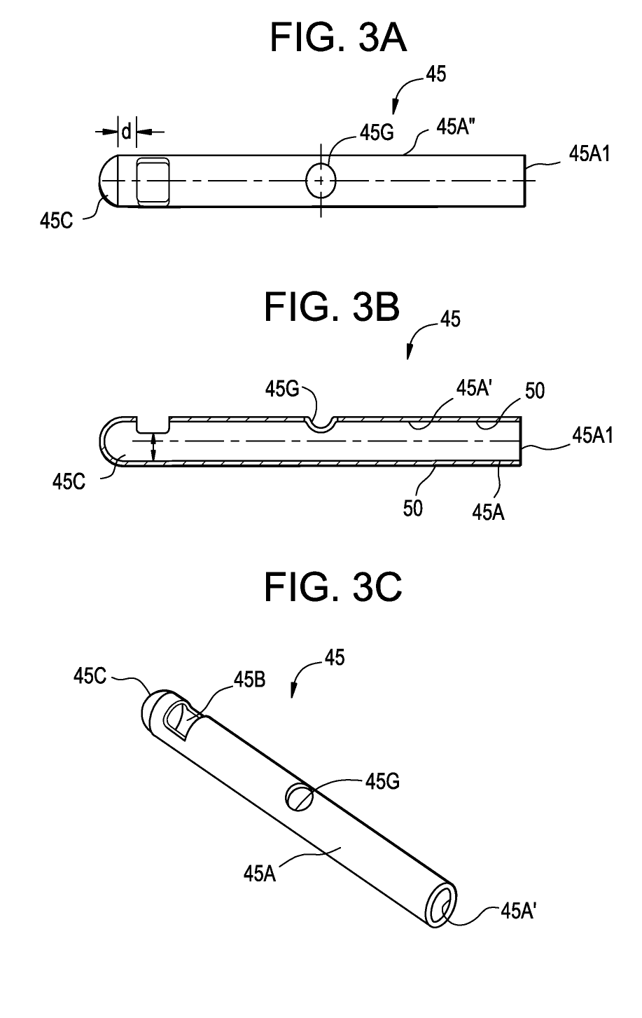

With reference to FIGS. 2A-3C a housing 45 for optical probe component 20 includes: a tubular body 45A having a first end 45A₁, a second end 45A₂, an inner surface 45A′, and an outer surface 45A″. A window (or window opening) 45B is formed in the tubular body 45A and is completely framed by a portion of the tubular body 45A. The window 45B of the housing 45 is displaced or off-set from the second end 45A₂ of the tubular body, preferably by a distance d of at least 0.2 mm, preferably by at least 0.5 mm, for example by at least 1 mm. Typically, 0.3 mm<d<2 mm. In some embodiments d>2 mm. That is, the edge of the window 45B does not extend all the way to the second end 45A₂ of the tubular body 45A. The window 45B has a width w where, for example, 0.05 mm<w<10 mm, preferably 0.05 mm<w<2.5 mm (e.g., 0.5 mm to 2 mm). The window 45B will be utilized as the exit window for the light beam that will be focused on tissues by the micro optic component 20.

The window 45B transmits light from the OCT probe component 20 to the tissues under observation, preferably at an angle 70° to 90° relative the optic axis of the OCT probe component 20 (i.e., relative to the optical axis of the fiber core), and allows scattered light to be transmitted back to the OCT probe component 20. In the embodiments shown in FIGS. 2A-3C, the window 45B is a perforation in the tubular housing 45. The dimensions provided in the exemplary embodiments shown FIGS. 2A, 2B, 3A and 3C are in mm.

The tubular body 45A may also include an aperture or a hole 45G to enable provision of adhesive into the tubular body 45A. The embodiment shown FIGS. 3A-3C also utilizes an end cap 45C that seals the end 45A₂ of the tubular body 45A.

Preferably, the outer surface 45A″ of the tubular body 45A is smooth and relatively slippery. A smooth tubular body 45A will have less friction with the inner lumen 48 or other tube in which is slides. Preferably, the tubular body 45A has a bore with a smooth surface 45A′ characterized by RMS surface roughness of a few microns, and more preferably RMS surface roughness in sub-micron range. Preferably the tubular body 45A has at least one low friction coating 50 (for example on its outer most surface 45A″) with coefficient of friction ≦0.3, more preferably with coefficient of friction ≦0.2.

In some embodiments, the tubular body 45A is stainless steel, and has a bore, and the surface 45A″ of the bore is polished (e.g., electro-polished) to the required smoothness. in some embodiments it is heat treated to eliminate impurities and burrs (if any are present), As stated above, in some embodiments, the surface 45A′ of the bore may contain a coating 50 to provide the required smoothness. According to some embodiments the outer surface 45A″ of the tubular body 45A has a coating 50 to provide the required smoothness. For example, the outer most layer of the tubular body 45A may have a coefficient of friction less than 0.3, and preferably less than 0.2. Coating 50 said coating includes Some examples of material options for such coatings 50 are: PVC, Hytrel, Nylon, Liquid Crystal Polymer Coatings, Teflon, low friction (typically fluoroalky silanes such as eptadecafluorotetrahydrodecyltrichlorosilane, as well as Dow Corning fluoroether silanes, DC2634, DC2604). Silane surface treatments and other silicone coatings can be applied to the surfaces as a thin coatings, or surface treatment on the order of monolayers to hundreds of nanometers thick, or thicker (micron range) if necessary. There are several advantages to utilizing coatings 50. For example, the coatings 50 can be applied on structural components like the (preferably steel) housing 45 to minimize the frictional forces with other OCT probe components, and provide better performance. For packaging and mechanical structural reasons, it is preferable to use metal (e.g., steel) housing 45 to house the OCT probe component 20 For example, the low friction outer layer or the coating 50 of the tubular body 45A can be obtained by coating micron/ sub-micron coatings of Teflon or Fluro-silane polymers on the surface 45A″. A low friction coating 50 can also be obtained by filling UV coating materials with micron sized beads of Teflon etc. Note that the low friction coatings 50 can also be applied to the torque tube or another power transmitting/rotation component 30.

Coating 50 Example(s)

Typical Teflon AF coating solution preparation: Teflon® AF (DuPont™ 1% in a fluoroether solvent, FC 40) is combined with a solution of adhesion binder (1 wt % in HFE7200) to produce a solution that is 1 wt % total in polymer mass. The solution is filtered through a coarse paper filter before use.

Exemplary coating and curing conditions: The housing 45 is cleaned by wiping with ethanol soaked kimwipe and dried thoroughly prior to use to remove organic contaminants on the surface. The coating is applied to the metal tubular body 45A through immersion into the coating solution or by other application method (contact transfer, spray coating, etc.). The coated part is cured in an oven, ramping up from 100 degrees to 165 degrees at 5 degrees/min, holding at 165° C. for 15 minutes. Then the temperature is ramped to 280° C. at 5 degrees/minute, holding the coated part at 280° C. for 60 minutes.

Exemplary silane coating and curing conditions: 0.5% solution of heptadecafluorotetrahydrododecyltrichlorosilane (Gelest, Morrisville, Pa.) is prepared by combining the perfluorosilane with anhydrous heptane. In this embodiment, the metal (e.g., steel) tubular body 45A is cleaned by wiping with an ethanol soaked kimwipe and dried thoroughly prior to use. The tubular body 45A is immersed in the coating solution, allowed to sit for 1 minute and, upon removal, is rinsed with heptane followed by ethanol.

Adhesion binder preparation and details are described, for example in: US published application, US20120189843.

With reference to FIGS. 1, 4A, 4B and 5A, 5B according to at least one embodiment, an OCT probe assembly 10 includes:

(i) a unitary micro optic component 20 having; (a) a light transmissive rod 25A having a first end 25A′, a second end 25A″, and a central axis 25S_{CA}; (b) a surface 25B situated on the second end and slanted with respect to the central axis 25_{CA}, wherein surface 25B is preferably a TIR (total internal reflectance) surface; (c) a lens element 25C situated on the rod 25A and being integral there to, and adjacent to the second end and to the 25B, the lens element 25C having a curved refractive surface 25C′ (in some embodiments the refractive surface 25C′ has at least one radius of curvature r1, where 100 μm≦r1≦5000 μm (and in some embodiments. 300 μm≧r1≧1000 μm) and a lens element 25C has a thickness t, where preferably 100 μm>t≧3000 μm (for example, t may be 100 μm, 200 μmm, 300 μm, 500 μm, 750 μm, 1000 μm, 2000 μm, or therebetween); and

(ii) a housing 45 surrounding the micro optic component 20; the housing 45 having: (a) a tubular body 45A (see, for example, FIGS. 2A-3C) with an entrance aperture 45D, and a window 45B formed in the tubular body 45A and completely framed by a portion of the tubular body (i.e., the opening in the tubular body is surrounded by the tubular body material, and may be formed as un uncovered hole or slot, or may be covered by a transparent material such as glass or plastic), the window 45B is situated over the lens element 25C (at least in at least some embodiments the window has a width w, where 0.5 mm<w<8 mm (for example 0.05 mm<w<2.5 mm, 0.1 mm<w<2 mm, or 0.2 mm<w<2 mm and preferably 1.7r1≦w≦2.2r1, for example 50 μm<w<2000 μm); and
(b) a seal or a plug situated on the end 45A₂ of the tubular body 45A, proximate to the window 45B.

In some embodiments, the second end 45A₂ is covered by a rounded cap (or plug) 45C, as shown, for example, in FIGS. 3A-3C. Preferably, the lens surface 25C′ is torroidal—i.e., preferably the lens surface 25C′ has two different radii of curvature r1, r2 to compensate for the astigmatism introduced by the cylindrical shape of the inner lumen, where r1 is not equal r2. Preferably 100 μm≦r1≦5000 μm and 100 μm≦r2≦5000 μm (and in some embodiments. 300 μm≦r1>1000 μm; 300 μm≦r2≦1000 μm).

As stated above, in these embodiments, preferably the micro optic component 20 is a unitary component, that is, it is a single component. For example, the micro optic component 20, including the rod 25A, the slanted surface 25B, and the lens element 25C, are made from the same optically transparent material. The micro optic component 20 can be molded, for example, as one unitary component of glass or plastic, or machined from the same glass body.

According to some embodiments, the OCT probe assembly 10 includes further includes a fiber mount 27 and an optical fiber 21 supported by the fiber mount 27. Fiber 21 can be a single mode fiber, for example SMF-28e®, available from Corning Incorporated, of Corning, N.Y. The mount 27 is located adjacent to the rod 25A, the optical fiber 21 is optically coupled to the rod 25A and the housing 45 surrounds the fiber mount 27 and at least a portion of the optical fiber 21 is supported therein. The fiber 21 may be in physical contact with the rod 25A or may be separated from it by a small air gap. An index matching material may be present in the space between the fiber 21 and the rod 25A. According to some embodiments, optical adhesive 49 is situated between the tubular body 45A and the fiber mount 27 and effectively seals the area between the inner surface 45A′ of the tubular body (i.e., the surface of the bore) and the micro optic component 20.

For example, according to some embodiments, OCT probe assembly 10 includes:

(i) a unitary micro optic component 20 including
(a) a light transmissive rod 25A having a first end 25A′, a second end 25A″, and a central axis 25A_{CA};
(b) a slanted surface 25B situated on the second end, wherein surface 25B is slanted with respect to the central axis 25A_{CA}, surface 25B is a reflective surface, for example a TIR (total internal reflection) surface, or a surface with a reflective coating thereon;
(c) a lens element 25C situated on the rod 25A and being integral there to, and adjacent to the second end and to the slanted surface 25B, the lens element 25C being preferably a raised lens element having a curved refractive surface 25C′ (for example, with at least one radius of curvature r1 wherein 100 μm≦r1≦5000 μm and a thickness t, where 100 μm≦t≦3000 μm (e.g., 250 μm≦w≦650 μm));
(d) a fiber mount 27 adjacent to the rod 25A and formed integral therewith, and

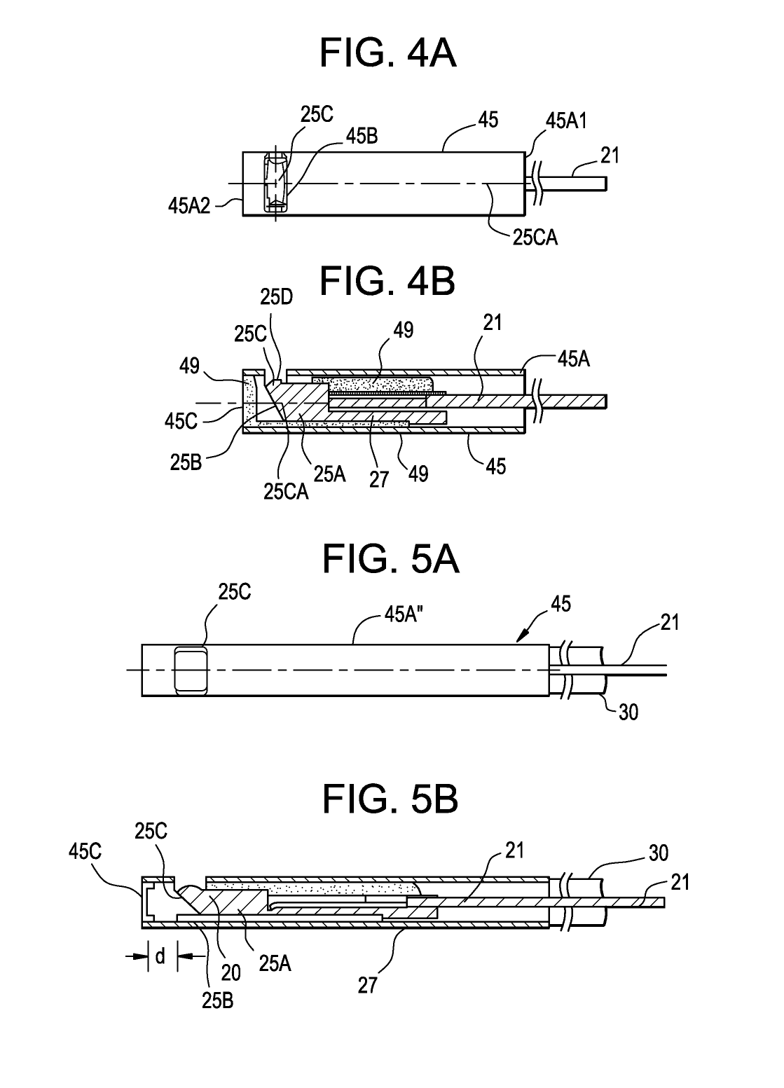

(ii) a housing 45 (see, for example, FIGS. 4 and 5) surrounding the micro optic component 20; the housing 45 having
(a) a tubular body 45A(see, for example, FIGS. 4 and 5) having a first end 45A₁, a second end 45A₂, an inner surface 45A′, and an outer surface 45A″, and
(b) a window 45B formed in the tubular body 45A and framed by a portion of the tubular body 45A, wherein the window 45B is situated over the lens element 25C and has a width w (for example, where 0.05 mm≦w≦10 mm (e.g., 0.05 mm to 5 mm, 0.1 mm to 2.5 mm, 0.2 mm to 2.5 mm, or 0.2 mm to 2 mm);
(c) a plug 45C that seals one end of the tubular body 45A, such that liquids, or unwanted particulates cannot enter through the aperture 45E at the end 45A₂ of the tubular body 45A to contact with the reflective surface 25B.

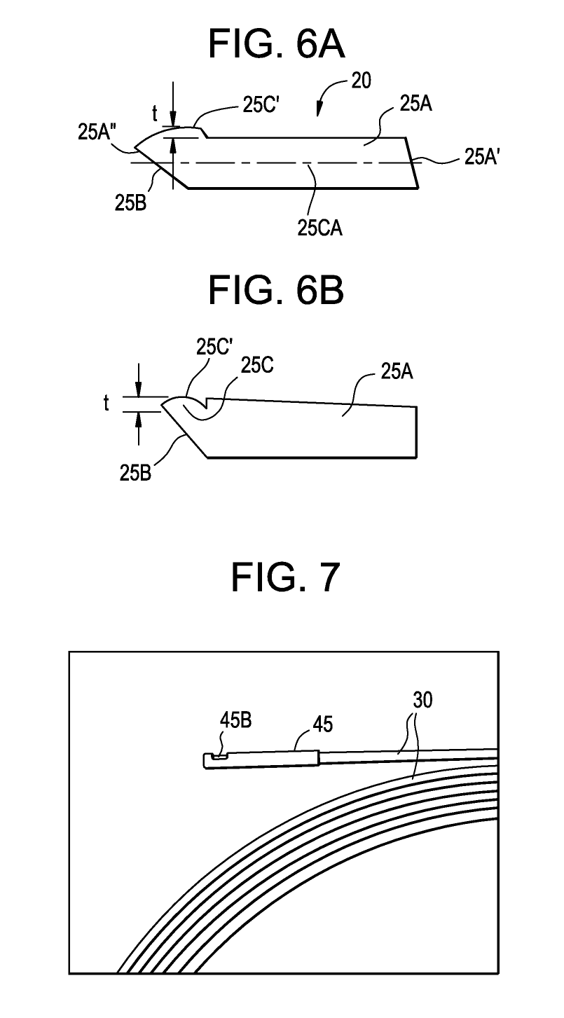

Some examples of the embodiments of the lens element 25C are illustrated in FIG. 6A and 6B. In FIG. 6A and 6B embodiments the lens elements 25C are integral to the optical probe component 20—i.e., in these embodiments the lens element 25C are made from the same material as the rest of the optical probe component 20,—they are not made as two different components that were cemented to one another.

As stated above, the fiber mount 27 may be made integral with the micro optic component 20. The fiber mount 27 may include a v-grove or a bore to support the fiber 21. Thus, in this embodiment, the micro optic component 20 (including the lens 20A, the rod 20A, the slanted surface 20C) and the fiber mount 27 are made from the same material. It can be molded, for example, as one unitary single component of glass or plastic, or made otherwise from the same optically transparent material (example, diamond turned glass). In at least some embodiments surface 25B is a reflective surface, such as TIR (total internal reflection) surface. It is noted that if the housing 45 did not contain a plug or a seal at the end 45A₂, contaminants could contact the TIR surface, thus disrupting light reflection in contact areas.

In some embodiments, a method of making an OCT probe assembly 10 comprises at least the following steps:

(i) providing a unitary micro optic component 20 (including a lens element and a light transmissive rod) coupled to the optical fiber 21, preferably the unitary micro optic component 20 also includes a fiber mount 27 (which is integral thereto) and the fiber 21 is situated on the fiber mount 27;

(ii) providing a housing 45 (see, for example, FIGS. 4 and 5) having a tubular body 45A with a bore situated therein, and a window 45B formed in the tubular body;

(iii) inserting the unitary micro optic component 20 and the fiber 21 into a housing 45, such that the micro optic component 20 and the fiber 21 enters one of the apertures 45D, 45E of the tubular body 45A, and is slid through at least a portion of the bore;

(iii) applying an adhesive material 49 through the window 45B into the bore;

(iv) sliding the micro optic component 20 inside the bore such that
(a) the motion of the micro optic component 20 spreads the adhesive material between the optic component 20 and the bore surface, thus at least filling with the adhesive material 49 the void situated between at least a portion of surface of the micro optic component 20 and the surface 45A′ of the bore, and
(b) the lens element is positioned in the window 45B; and

(v) preferably sealing or preferably plugging the second aperture 45E of the tubular body (e.g., forming a plug 45C with an adhesive, or attaching a plug 45C to the second end 45A₂ over the second aperture 45E of the tubular body 45A).

Although in some embodiments the fiber mount can be made separate and then be attached to the unitary micro optic component 20 that includs a lens element, a light transmissive rod and the slanted surface (e.g. a TIR surface), preferably the unitary micro optic component 20 according to at least some of the embodiments described herein also includes the fiber mount 27 that is made integrally therewith. In these embodiments the fiber mount 27 is made from the same optically transparent material as the rest of the micro optic component 20. For example, for example the unitary micro optic component 20 including the lens element, the light transmissive rod, the slanted surface and the fiber mount 27 can be molded as one unit from the same plastic or glass. Alternatively, also for example, it can be micromachined, from glass or plastic. For example, the unitary micro optic component 20 including the fiber mount 27 can be dimond-turned from the same glass body.

According to some embodiments additional adhesive is supplied through the hole 45G (glue hole). The hole 45G is situated in the tubular body 45A, and one embodiment is illustrated in FIGS. 3A-3C.

In some embodiments, a method of making an OCT probe assembly 10 comprises at least the following steps:

(i) providing a unitary micro optic component 20 (including a lens element and a light transmissive rod) with a with a fiber mount 27 integral thereto;

(ii) providing a housing 45 (see, for example, FIGS. 4 and 5) having a tubular body 45A with a bore situated therein, and a window 45B formed in the tubular body;

(iii) inserting the unitary micro optic component 20 into a housing 45, such that the micro optic component 20 enters entrance aperture 45D of the tubular body 45A, and is slid through the bore such the lens element exits a second aperture 45E of the tubular body 45A;

(iii) applying an adhesive material 49 through the window 45B into the bore;

(iv) sliding the micro optic component 20 back through the second aperture 45E toward the entrance aperture 45D such that the motion of the micro optic component 20 spreads the adhesive material between the optic component 20 and the bore surface, thus at least filling with the adhesive material 49 the void situated between at least a portion of surface of the micro optic component 20 and the surface 45A′ of the bore, until the lens element is positioned in the window 45B; and

(v) preferably, sealing or plugging the second aperture 45E of the tubular body (e.g., forming a plug 45C with an adhesive, or attaching a plug 45C to the second end 45A₂ over the second aperture 45E of the tubular body 45A).

In some embodiments, a method of making an OCT probe assembly 10 comprises at least the following steps:

(i) providing a unitary micro optic component 20 (including a lens element and a light transmissive rod) with a with a fiber mount 27 integral thereto;

(ii) providing a housing 45 (see, for example, FIGS. 4 and 5) having a tubular body 45A with a bore situated therein, and a window 45B formed in the tubular body;

(iii) inserting the unitary micro optic component 20 into a housing 45, such that the micro optic component 20 enters aperture 45E of the tubular body 45A, and is slid through the bore;

(iii) applying an adhesive material 49 through the window 45B into the bore;

(iv) sliding the micro optic component 20 within the bore such that the motion of the micro optic component 20 spreads the adhesive material between the optic component 20 and the bore surface, thus at least filling with the adhesive material 49 the void situated between at least a portion of surface of the micro optic component 20 and the surface 45A′ of the bore, and positioning the lens element 25C in (i.e., inside) the window 45B; and

(v) preferably, sealing or plugging the second aperture 45E of the tubular body (e.g., forming a plug 45C with an adhesive, or attaching a plug 45C to the second end 45A₂ over the second aperture 45E of the tubular body 45A).

In at least one embodiment, a method of making an OCT probe assembly 10 comprises at least the following steps:

(i) providing a unitary micro optic component 20 including
(a) a light transmissive rod 25A having a first end 25A′, a second end 25A″, and a central axis 25A_{CA};
(b) a reflective surface 25B situated on the second end and slanted with respect to the central axis;
(c) a raised lens element 25C situated on the rod 25A and integral thereto, the raised lens element 25C being adjacent to the second end 45A₂ and to the reflective surface 25B, the lens element 25C having a curved refractive surface 25C′;
(d) a fiber mount 27 adjacent to the rod 25A and formed integral thereto

(ii) providing a housing 45 (see, for example, FIGS. 4 and 5) having a tubular body 45A with a bore situated therein, and a window 45B formed in the tubular body;

(iii) inserting the unitary micro optic component 20 into a housing 45, such that the micro optic component 20 enters entrance aperture 45D of the tubular body 45, and is slid through the bore such that at least the reflective surface 25B, the lens element 25C and the rod 25A exits a second aperture 45E of the tubular body 45A;

(iv) applying an adhesive material 49 through the window 45B into the bore;

(v) sliding the micro optic component 20 back through the second aperture 45E toward the entrance aperture 45D such that the motion of the micro optic component 20 spreads the adhesive material between the optic component 20 and the bore surface, thus at least filling with the adhesive material 49 the void situated between at least a portion of surface of the micro optic component 20 and the surface 45A″ of the bore;

(v) positioning the lens element 25C inside the window 45B; and
(vi) sealing the second aperture 45E of the tubular body 45A (e.g., forming a plug 45C with the adhesive 49 on the end 45A₂ of the tubular body).

The plug 45C seals one end of the tubular body 45A, such that liquids, or unwanted particulates cannot enter through the aperture 45E at the end 45A₂ of the tubular body 45A to contact the reflective surface 25B.

For example, according to some embodiments, during assembly, the fiber 21 and the optical probe component 20 is inserted into the tubular body 45A, (which, in some embodiments, is made of steel) and then secured into place by the application of a UV and/or heat curing adhesive. Since both ends 45A₁, 45A₂ of the tubular body 45A are still open during this step of the assembly process, the tubular body 45A can be preloaded with the fiber 21/optical probe component 20 during the assembly of the OCT probe 10. The optical probe component 20 (with the fiber 21 attached or coupled thereto) is carefully pulled back into the tubular body 45A towards the end 45A₁. That is, the fiber is attached to the fiber mount that is an integral part of the probe component 20, and is inserted into the tubular body 45A through either aperture 45D or 45E. When the end of the probe component 20 that is proximate to the fiber is aligned with the window 45B, a UV/thermal curable adhesive 49 is applied through the window 45B of the tubular body 45A onto the fiber mount 27 or the fiber mount portion of the probe component 20. More adhesive is applied as the optical probe component 20 is pulled towards the end 45A₁. More specifically, the adhesive 49 has a high viscosity (greater than 1250 (CPs) and preferably less than 2500 (CPs) at 23° C. at 100 RPM) which minimizes the chance of the adhesive flowing into the void from one end of the tubular body to the other end of the tubular body 45A. Preferably the adhesive's viscosity is greater than 1300 (CPs) and less than 2000 (CPs) at 23° C. at 100 RPM. In some embodiments, the adhesive 49 has viscosity greater than 1400 (CPs) at 23° C. at 100 RPM, and in some embodiments greater than 1500 (CPs) at 23° C. at 100 RPM. For example a UV curable epoxy with Viscosity of 1765 (CPs) at 23° C. at 100 RPM such as EPO-TEK® 0G198-55available from Fiber Optic Center, Inc of New Bedford, Mass. Enough adhesive is applied such that the height of the adhesive is approximately the same height as the outer diameter of the tubular body 45A. That is, the high viscosity or thixotropic nature of the adhesive inhibits the adhesive from flowing. For example, adhesive 49 is piled on top of itself to fill the area between the top of the probe component to the inner surface of the tubular body. The fiber 21/optical probe component 20 subassembly is then slowly pulled into the tubular body 45A, which spreads the adhesive along the length of the probe and into the tubular body 45A. More adhesive 49 is applied, as the probe component 20 is pulled in towards the end 45A₁, such that there is always contact between the adhesive 49 and the inner diameter (surface 45A′) of the tubular body 45A. Once the optical probe component 20 has been pulled into its final position, such that the lens surface 25C′ is aligned with the window 45B, UV light cures the adhesive 49 in position. Then, preferably, the OCT probe assembly 10 is subjected to thermal cure, which cures the adhesive 49 that is not cured with exposure to a UV source. After this step, the open end of the tubular body 45A adjacent to the TIR surface is sealed off either by adhesive 49 (as shown, for example, in FIG. 4B) or by inserting an end cap 45C as shown for example in FIG. 5B).

For example, the probe component 20 and the optical fiber coupled or attached thereto can be either inserted into the tubular body 45A lens first,. Alternatively the tubular body 45A can be preloaded onto the fiber 21 prior to assembling coupling the fiber to the probe component 20, and the then the fiber can be mounted on the fiber mount 27, and adhered to it such that it is optically coupled to the transmissive rod 25A of the probe component 20, forming fiber/probe component subassembly. Preloading the tubular body 45A onto the fiber 21 enables the fiber/probe component subassembly to be pulled into the bore of the tubular body 45A with the fiber end of the fiber/probe component subassembly entering the tubular body 45A first, which reduces the possibility of damage to the lens surface 25C′ of the lens element 25C. Pulling the fiber/probe component subassembly back into the tubular body 45A towards the end 45A₁ also enable the optimum application of adhesive 49. The back end of the probe component 20 (i.e., the end closest to the fiber) is pulled into the t tubular body 45A and rotated until the flat portion of the probe component 20 (fiber mount 27) where the fiber was previously glued into place is facing up and until the end of the probe component 20 is even with the edge of the window 45B in the tubular body 45A. An adhesive (which is thixotropic) is applied through the window 45B onto the probe in such a way as to fill the window 45B with the adhesive to where the height of the adhesive is approximately the same height as the outer diameter of the tubular body. The probe is then slowly pulled into the tube while continuing to apply adhesive as the probe is pulled into the tube , such that there is always contact between the adhesive 49 and the inner diameter (surface 45A′) of the tubular body. Once the optical probe component 20 has been pulled into its final position, such that the lens surface 25C′ is aligned with the window 45B. The adhesive 49 is then is cured in position.

After this step, the open end of the tubular body 45A adjacent to the TIR surface is sealed off either by adhesive 49 (as shown, for example, in FIG. 4B) or by inserting an end cap 45C as shown for example in FIG. 5B).

Alternatively, an extra aperture (or hole) 45G in the tubular body 45A can be utilized (FIG. 3A-3C) to providing adhesive 49 into the bore. This approach may reduce the likelihood of contaminating the lens element with an adhesive. The embodiment shown FIGS. 3A-3C also utilizes the end cap 45C (that acts as a pre-seal), thus no additional sealing at the second end 45A₂ of the tubular body 45A may be required. This approach reduces processing steps while simultaneously providing low risk for contamination on lens surface 25C′.

According to some embodiments a torque tube or another power transmitting/rotation component 30 is attached to the fiber mount 27 and/or to the optical fiber 21, for rotating and translating the micro optic component 20 within the body during scanning. According to at least some embodiments a part of the torque tube or of the power transmitting/rotation component is inserted inside the bore of the housing 45. Accordingly, it is preferable that during the steps of insertion of the micro optic component 20 inside the housing 45, application an adhesive material through the window 45B into the bore, and sliding the micro optic component 20 back through the second aperture 45E towards the first aperture 45D, one does not deposit adhesive in the portion of bore that is intended to receive the torque tube (or another power transmitting/rotation component) 30.

FIG. 6 illustrates a typical stainless steel coiled wire torque tube 30 attached to the OCT probe assembly 10, as utilized in some exemplary embodiments OCT probes 5. More specifically, FIG. 6 illustrates both the stainless steel coiled wire torque tube 30 and the housing 45. In this embodiment, the stainless steel coiled wire torque tube 30 includes multi-coil stainless steel spring with precise dimensional control. An optical fiber 21 is inserted into the torque tube 30 so that the torque tube 30 surrounds the fiber 21. Generally, stainless steel coiled wire torque torque tube comprises of three or more spring coils, with at least two of the spring coils wound in clockwise or counter clockwise direction and at least one other spring coils wound in the opposite direction.

The optical probe component 20, the optical fiber 21, the torque tube or another power transmitting/rotation component 30 surrounding this optical fiber 21 and the tubular housing 45 are threaded through a closely fitting transparent polymer tube or the inner lumen 48, to form OCT probe 5.

### Example

1

housing 45.

In this exemplary embodiment the tubular body 45A of the housing 45 is cut from a long tube that has appropriate dimensions, smoothness and roundness, and that is made of an appropriate material such as stainless steel. For some exemplary embodiments, the inner diameter of the tubular body 45A is preferably about 1 mm and the outside diameter is about 1.3 mm For example, the long tube is selected to be round and to have the outside surface that is relatively smooth with a surface roughness of a few tens of microns (e.g., <50 μm) or better (e.g., <10 μm). The long tube is cut several times to the required length, in order to provide the needed numbers of the tubular bodies 45A. The cutting process can be, for example, a dicing process, a wire sawing process, or preferably EDM (electric discharge machining) process. If a dicing or a wire sawing process, care has to be taken to remove any sharp edges(i.e., and the tubular body 45A is deburred). Without this process, these sharp edges may damage the inner lumen or polymer tubular body 48 in which the OCT probe assembly is inserted and which will be rotating inside the inner lumen. A dicing process or EDM process can also be used for making the window in the tubular body. Again, it is preferable to round out the sharp edges and remove any burr material left.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

It will be apparent to those skilled in the art that various modifications and variations can be made without departing from the spirit or scope of the invention. Since modifications combinations, sub-combinations and variations of the disclosed embodiments incorporating the spirit and substance of the invention may occur to persons skilled in the art, the invention should be construed to include everything within the scope of the appended claims and their equivalents.

What is claimed is:

1. A housing for the OCT probe component comprising:
(a) a tubular body with an inner diameter of less than 5 mm, a first end, a second end; and
(b) a window formed in the tubular body closer to said second end than to said first end, displaced from said second end, and framed by a portion of the tubular body, wherein the window has a width w.

2. The housing for the OCT probe component according to claim 1, wherein 0.05 mm<w<8 mm; or said window is displaced from said second end by a distance d of at least 0.2 mm.

3. The housing for the OCT probe component according to claim 2, wherein 50 μm<w<2500 μm.

4. The housing for the OCT probe component according to claim 1, wherein said tubular body includes at least one surface that has RMS surface roughness of ≦5 μm;
or (ii) the tubular body has at least one surface with the coefficient of friction ≦0.3.

5. The housing for the OCT probe component according to claim 1, wherein the tubular body has at least one low friction coating with the coefficient of friction ≦0.2.

6. The housing for the OCT probe component according to claim 1, wherein the tubular body with an inner diameter of not greater than 1.5 mm.

7. An OCT probe assembly comprising:
(i) a unitary micro optic component including
(a) a light transmissive rod having a first end, a second end, and a central axis;
(b) a reflective surface situated on the second end and slanted with respect to the central axis;
(c) a lens element situated on the rod and being integral thereto, the lens element being adjacent to the second end and to the reflective surface, the lens element having a curved refractive surface with at least one radius of curvature r1 wherein
100 μm<r1<5000 μm; and
(ii) a housing surrounding the micro optic component; the housing having
(c) a tubular body with an end adjacent to said second end of said rod, and
(d) a window formed in the tubular body and framed by a portion of the tubular body, wherein the window is situated over the lens element and has a width w, where 50 μm<w<2500 μm.

8. The OCT probe assembly according to claim 7, wherein the lens element is a raised lens element with a thickness t, and t is 100 μm<t<3000 μm.

9. The OCT probe assembly according to claim 7, further comprising an optical fiber optically coupled to the rod, wherein the tubular body surrounds at least a portion of the optical fiber.

10. The OCT probe assembly according to claim 7, further comprising:
(a) a fiber mount,
(b) an optical fiber supported by the fiber mount, the fiber mount being positioned adjacent to the first end of said rod, the optical fiber having an output end optically coupled to the first end of said rod,
wherein the tubular body surrounds the fiber mount and at least a portion of the optical fiber supported therein.

11. The OCT probe assembly according to claim 10, wherein said tubular body includes an aperture situated in said tubular body, the aperture being spaced apart from the window.

12. The OCT probe assembly of claim 10, further comprising:
optical adhesive situated between the tubular body and:
(i) the fiber mount; and/or
(ii) the optical fiber.

13. The OCT probe assembly of claim 10, wherein said housing further comprises: a rounded cap situated proximate to the second end of said rod.

14. The OCT probe assembly of claim 13, wherein the rounded cap is situated on the end of the tubular body proximate to the reflective surface.

15. The OCT probe assembly of claim 11, further comprising: an adhesive sealant sealing the end of the tubular body proximate said second end of said rod, wherein the sealant is not in contact with the reflective surface.

16. An OCT probe assembly comprising:
(i) a unitary micro optic component including
(a) a light transmissive rod having a first and a second end and a central axis
(b) a reflective surface situated on the second end and slanted with respect to the central axis
(c) a raised lens element situated on the rod and being integral there to, and adjacent to the second end and to the reflective surface, the lens element having a curved refractive surface with at least one radius of curvature r1, wherein
100 μm<r1<5000 μm and a thickness t, where 100 μm<t<3000 μm;
(d) a fiber mount adjacent to the rod and formed integral thereto, and
(ii) a housing of claim 1 surrounding the micro optic component wherein the
(a) a window formed in the tubular body and framed by a portion of the tubular body, wherein the window is situated over the lens element and (i) has a width w, where 8 mm>w>0.05 mm; and/or (ii); or said is displaced from said second end by a distance d of at least 0.2 mm.

17. The OCT probe assembly according to claim 16, further comprising an optical fiber supported by the fiber mount and optically coupled to the rod, wherein the tubular body surrounds at least a portion of the optical fiber.

18. The OCT probe assembly according to claim 16, wherein tubular body has (i) at least one smooth surface characterized by RMS surface roughness of ≦5 μm; and/or (ii) has at least one low friction coating with coefficient of friction ≦0.2.

19. A method of making an OCT probe assembly comprising:
(i) providing a unitary micro optic component including
(a) a light transmissive rod having a first and a second end and a central axis;
(b) a reflective surface situated on the second end and slanted with respect to the central axis;
(c) a raised lens element situated on the rod and being integral thereto, and adjacent to the second end and to the reflective surface, the lens element having a curved refractive surface
(d) a fiber mount adjacent to the rod and formed integral thereto'
(ii) inserting the unitary micro optic component into a housing having a tubular body with a bore, and a window formed in the tubular body and framed by a portion of the tubular body, such that the micro optic component enters an entrance aperture side of the tubular body, and is slid through the bore such that at least the reflective surface and the lens element exit a second aperture of the tubular body;
(iii) applying an adhesive material through the window into the bore;
(vii) sliding the micro optic component back through the second aperture toward the first aperture such that (i) the motion of the micro optic component spreads the glue between the optic component and the bore surface, thus at least filling a void between the at least a portion of at least one surface of the micro optic component and the bore surface;
(viii) positioning the lens element in the window; and
(ix) sealing or closing the second aperture of the tubular body.

20. An OCT probe assembly comprising:
(i) a micro optic component including
(a) a light transmissive rod having a first end, a second end, and a central axis;
(b) a reflective surface situated on the second end and slanted with respect to the central axis;
(c) a refractive surface adjacent to the second end; and
(ii) a housing surrounding the micro optic component; the housing having
a) a tubular body, and
b) a window formed in the tubular body and situated at least 0.2 mm away from end of the tubular body nearest the window, the window being framed by a portion of the tubular body and is situated over the refractive surface;
wherein a tubular body has a smooth outer and/or an inner surface characterized by (i) RMS surface roughness of ≦2 μm, or has at least one surface with coefficient of friction ≦0.2.
